(19) United States
(12) Patent Application Publication
(10) Pub. No.: US 2023/0241454 A1
(43) Pub. Date: Aug. 3, 2023

(54) MULTI-INPUT AUTOMATIC MONITORING OF MOTION TRACKING SYSTEM AND ACTUATION

(71) Applicant: SWORD HEALTH, S.A., Porto (PT)

(72) Inventors: José Carlos COELHO ALVES, Porto (PT); Diego Ricardo CASTRO GONÇALVES, Porto (PT); Márcio Filipe MOUTINHO COLUNAS, Porto (PT); Ana Clara FERREIRA MATOS, Porto (PT); Luís Ungaro PINTO COELHO, Porto (PT); Pedro Henrique OLIVEIRA SANTOS, Porto (PT); Luís António CORREIA DE OLIVEIRA, Porto (PT); Pedro Filipe Xavier RODRIGUEAS, Porto (PT); Virgílio António FERRO BENTO, Porto (PT)

(21) Appl. No.: 18/128,197

(22) Filed: Mar. 29, 2023

Related U.S. Application Data
(63) Continuation of application No. PCT/EP2022/052707, filed on Feb. 4, 2022.

(30) Foreign Application Priority Data
Feb. 4, 2021 (EP) 21398002.2

(51) Int. Cl. A63B 24/00; G09B 19/00; G16H 20/30; G16H 40/67

(52) U.S. Cl. CPC A63B 24/0062; G09B 19/003; A63B 24/0075; G16H 20/30; G16H 40/67; A61B 5/4824

(57) ABSTRACT

Methods for automatically monitoring operation of a motion tracking system and actuating based on the operation, the motion tracking system comprising both a computing device, and a plurality of inertial measurement units adapted to be arranged on a body of a user. The computing device processes measurements of the units and sound captured by at least one microphone to provide flags related to the motion tracking procedure with regards to the motion input and the sound input, and actuates when motion-related flags and sound-related flags have been provided.

### CROSS-REFERENCE TO RELATED APPLICATIONS

This application is a continuation of International Application No. PCT/EP2022/052707, filed internationally on Feb. 4, 2022, which claims priority to European Application No. 21398002.2, filed on Feb. 4, 2021, all of which are hereby incorporated by reference in their entireties.

### TECHNICAL FIELD

The present disclosure relates to the technical field of motion tracking systems. More particularly, the present disclosure relates to automatic supervision of the operation of a motion tracking system while a user thereof is performing physical exercises with inertial measurement units thereon, and automatic notification of the user and/or actuation of the system when the result of the supervision, which is of multiple inputs, is indicative of a problem with the system or the user thereof.

### BACKGROUND

In many physical rehabilitation procedures, a person has to exercise by moving body members in a certain way to recover the mobility of an injured part of the body or reduce the pain that the person suffers, for example by performing stretching movements. With these procedures, the user can recover from a hip injury, a shoulder injury, a neck injury, etc. more rapidly. The physical rehabilitation of people typically takes place at medical facilities and under supervision of a physical therapist or a doctor. Without said supervision, the person that rehabilitates runs the risk of getting injured even if she/he knows which movements she/he must perform because the person does not know whether all the movements and repetitions thereof are correctly reproduced. The need for medical facilities and availability of therapists or doctors limits the amount of people that can physically rehabilitate each day.

Motion tracking or motion capture of a target is a technical process used in many applications, such as, control of machines, automatization of processes, or gait analysis to name a few. The motion of a person or an object can be tracked by means of sensors that are attached to the person or object. The sensors, which are or include inertial measurement units, and the measurements thereof are indicative of the positions and angles of the tracked anatomical landmarks or body members; by processing these measurements, so establishment of the motion of the tracked landmarks or body members (e.g. the range of motion) can be achieved by processing the measurements.

With motion tracking systems, the physical rehabilitation of the person can be supervised accurately thanks to the measurements of the sensors. The measurements enable automated supervision but they do not necessarily reflect whether the person is experiencing physical condition problems or system problems.

In order to reduce the risk of the person getting injured and the supervision of a physical therapist or doctor, it is deemed necessary to have procedures for automatically supervising the motion tracking system used and the user thereof, and automatically making decisions on how to address problematic situations during use of the motion tracking system.

### SUMMARY

A first aspect of the disclosure relates to a method for automatically monitoring operation of a motion tracking system and actuating based on the operation, the motion tracking system comprising both a computing device, and a plurality of inertial measurement units adapted to be arranged on a body of a user, the computing device comprising both a data communications module and at least one microphone, the method comprising:

commanding, by the computing device, to provide one or more user perceptible signals indicative of at least one predetermined movement to be performed by the user;

providing, by each unit of the plurality of units, measurements of body members of the user both while each unit is arranged on a respective body member and after the user has been instructed to perform the at least one predetermined movement;

processing, by the computing device, the measurements of the plurality of units to provide a motion sequence, MS, of at least part of at least some body members of the user;

storing, by the computing device and associated with both the user and a movement of the at least one predetermined movement, the MS and/or a range of motion, ROM, of one or more of the some body members of the user, the ROM being computed by the computing device based on the MS;

processing, by the computing device, the MS and/or the ROM to check fulfillment thereof of one or more predetermined criteria associated with a respective movement of the at least one predetermined movement and counting, by the computing device, a number of times that each of the one or more predetermined criteria are unfulfilled;

providing, by the computing device, a motion-related flag each time a counted number of times does not meet a respective predetermined threshold;

processing, by the computing device, sound captured with the at least one microphone to detect human voice therein;

processing, by the computing device, the sound captured to detect human gasps therein based on the detected human voice;

providing, by the computing device, a sound-related flag when it determines that the user has gasped a number of times exceeding a respective predetermined threshold; and

actuating, by the computing device, when both at least one motion-related flag and at least one sound-related flag have been provided, and the step of actuating comprising at least one of the following:
commanding to provide one or more user perceptible signals indicative of the at least one motion-related flag provided and/or indicative of a corrective action to be performed by the user based on the at least one motion-related flag provided; and
communicating the at least one motion-related flag provided to one or more electronic devices.

The at least one motion-related flag indicates that the user may be experiencing some problems during performance of the movements, which typically are assigned to the user by a physical therapist or a doctor. To this end, the motion sequence, MS, and the range of motion, ROM, resulting from the processing of the measurements describe how the body members of the user move.

The users of motion tracking systems many times do not exercise to the best of their capabilities and underperform, thereby not rehabilitating their body as best as possible but without much risk of getting injured. However, when the user is not exercising correctly according to the predetermined criteria and she/he gasps during the rehabilitation many times, there is a high likelihood that the physical condition of the user is not good and immediate action or actions need be taken. This means that the at least one sound-related flag indicates that the user is feeling tired or has pain, and the existence of both sound-related flags and motion-related flags is used by the motion tracking system to automatically actuate.

The one or more predetermined criteria are set according to each particular predetermined movement that users of the motion tracking system may have to perform.

The storage of any or both of the MS and the ROM enables tracking the evolution of the rehabilitation of the patient over time. The evolution over a number of rehabilitation sessions can also be used to establish whether a user is experiencing problems while using the motion tracking system and, thus, provide motion-related flags. In this sense, the computing device may use as predetermined criterion or criteria the performance of the user in the past. A user that underperforms in comparison to previous performance thereof can evidence incorrect reproduction of predetermined movements or a physical condition worse than that in the past, both of which entail a risk of getting injured if the user is really tired or has a poor physical condition as revealed by the sound-related flags.

The actuation of the motion tracking system is triggered by the computing device. The actuation can be the communication to the one or more electronic devices of each motion-related flag provided; each motion-related flag preferably indicates which predetermined criterion or criteria have not been fulfilled and at which point in time they have not been fulfilled, and can also include data for identification of the user. The one or more electronic devices preferably are managed or are in data communications with devices of a physical therapist or doctor, hence said therapist or doctor is informed of the detected problems and, this way, she/he is able to reach out to the user when these situations occur. Additionally or alternatively, the actuation can be in the form of provision of information to the user, the information being about the detected problem or problems (with indications such as, but without limitation: the physical therapist is being contacted for assistance, the entire prescribed ROM not being reached by the user, user taking too much time to start each repetition of a predetermined movement, etc.), and/or about how the computing device considers that the problem(s) should be dealt with (with indications such as: take a break for some minutes, stop exercising, stretch, apply ice on joints, skip to the next predetermined movement, do not move the hip while flexing the arm, etc.).

In some embodiments, the step of actuating takes place when a time elapsed between the provision of the at least one sound-related flag and the provision of the at least one motion-related flag is less than a predetermined time threshold.

The probability of an existing problem is greater the less time has elapsed between the provision of at least one sound-related flag and the provision of at least one motion-related flag since usually there is a correspondence between the user gasping too much and not fulfilling predetermined criteria. Therefore, the actuation of the motion tracking system can be limited to the situations in which this time elapsed is less than the predetermined time threshold, e.g. two minutes, one minute, twenty seconds, etc.

In some embodiments, the one or more predetermined criteria comprise at least one (or some, or each) of: the MS not fulfilling one or more predetermined constraints set for each predetermined movement commanded; the ROM being lower than the ROM previously stored by the computing device for the same user and for the same predetermined movement; and an elapsed time for the one or more body members in the MS to be in the initial position of the predetermined movement commanded exceeding a predetermined time threshold set for each predetermined movement commanded.

The predetermined constraints numerically quantify thresholds or ranges of positions, angles, speeds and/or accelerations that particular body members should fulfill at two or more time instants during reproduction of the respective movement, thereby making possible to digitally validate the correct reproduction of the movement. The setting of these predetermined constraints for use in motion tracking systems is disclosed in international application no. PCT/EP2019/066237, which is incorporated by reference in its entirety herein. As disclosed in said international application, each predetermined movement has one or more predetermined constraints to be met in order to determine correct reproduction of a movement. The predetermined constraints preferably comprise one or more orientations in the form of angles, and/or one or more accelerations in the form of acceleration values. The concerned body members have to be beneath or above the respective angles or accelerations values in one moment or another during the performance of the movement.

The computed ROM for a current movement reproduced by the user can reveal underperformance of the user when compared to previous ROM or ROMs of the same user for the same movement, thus a difference between the current ROM and the previous ROM or ROMs is to be computed.

It can be appreciated that sometimes the user will attain ROMs greater than those requested from her/him during performance of the predetermined movement, for instance because the user is feeling well that day or the ROM requested from her/him is too low for the actual physical condition that the user is in. In a subsequent session, the user may not feel so good, the ROM not be exceeded and at the same time be below the previous ROMs, yet this situation is not one in which there is risk that the user will get injured, so even if the motion-related flag is provided, the sound-related flag may not be provided and no actuation is necessary.

The user normally is to make one or more repetitions of a predetermined movement because in physical rehabilitation each predetermined movement forms one or more series of a physical exercise. The time it takes the user to move her/his limbs to the initial position of the predetermined movement after having finished the same movement can be an indicator of how tired the user is, or how painful the predetermined movement is for the user. When the elapsed time exceeds a predetermined time threshold a number of times, a motion-related flag is provided.

Different predetermined movements usually have different predetermined thresholds, constraints or ROMs. By way of example, if one predetermined movement is a squat and another predetermined movement is from lying on the floor to stand and jump, the predetermined time threshold to go back to the initial position in the squat will normally be lower than that for the lying on the floor to stand and jump movement, for example five seconds and ten seconds, respectively.

In some embodiments, the one or more predetermined criteria at least comprises the predetermined criterion of the ROM being lower than the ROM previously stored, and said criterion comprises: an average of the ROM being lower than an average of the ROM previously stored, and a difference between the averages exceeding a predetermined average ROM threshold; and/or the ROM of the respective movement of the one or more predetermined movements in a plurality of repetitions of the respective movement being lower than the average of the ROM previously stored.

The ROMs can be compared either by way of the respective average values, or by computing how many repetitions of the predetermined movement have a ROM that is below the average of the ROM previously stored. At least in the first case, a predetermined average ROM threshold is set such that slightly lower ROMs do not result in the provision of the motion-related flag. By way of example, this predetermined average ROM threshold can be 2%, or 5%, and so when the average current ROM is lower than the average previous ROM by less than 2% or 5%, it is considered that the user is not underperforming.

In some embodiments, the step of actuating comprises the step of communicating, and the step of communicating the at least one motion-related flag further comprises communicating one or more of: the at least one sound-related flag, the MS, the ROM of a current activity session of the user of the motion tracking system, the MS previously stored, and the ROM previously stored (i.e. of previous activity sessions of the user).

Data generated by the computing device during the session of the user can be provided to the one or more electronic devices for informing the therapist or doctor of the evolution of the ongoing session and, optionally, of previous sessions. The data can be assessed by the therapist or doctor to better understand what can be the problem of the user that resulted in the provision of the motion-related and the sound-related flags. In this sense, the sound-related flag(s) can be transmitted too, which informs the therapist or doctor about what kind of problem there is, particularly when a method according to the second aspect of the disclosure is also conducted.

In some embodiments, the step of actuating comprises the step of commanding, and the one or more user perceptible signals indicative of the corrective action to be performed by the user comprise indications of an end of a motion tracking procedure when both one or more motion-related flags and one or more sound-related flags have been provided. In some of these embodiments, the one or more motion-related flags provided exceeds a predetermined motion-related flag threshold. In some of these embodiments, the one or more sound-related flags provided exceeds a predetermined sound-related flag threshold.

When no risk of injury can be accepted, for example but without limitation, when too many motion-related and/or sound-related flags have been provided, the user is instructed to stop the activity and not continue with the physical exercising.

In some of these embodiments, the indications of the end of the motion tracking procedure are provided in the form of the one or more user perceptible signals also based on data of the user and/or data of the predetermined movement last commanded to the user when the computing device performs the step of actuating.

The motion tracking procedure can be ended when the motion-related flags and sound-related flags have been provided, but also taking into account the particulars of the user of the motion tracking system, and/or the predetermined movement when the actuation was triggered.

By way of example, a user having some particular diseases or an elder person are more prone to injuries than other users. Medical data about the user is first registered in the motion tracking system or a server that is communicatively coupled with the motion tracking system, therefore when the conditions of the user are worse, the less demanding the computing device is to end the motion tracking procedure, i.e. the fewer motion-related flags and/or the fewer sound-related flags are necessary to halt the procedure.

Certain predetermined movements are more demanding than others. Each predetermined movement registered in the motion tracking system preferably has data associated therewith, the data comprising a quantitative indicator of the physical demand involved in the movement, or a formula that quantifies the physical demand based on the predetermined constraints associated therewith. By way of another example, when flags are provided in predetermined movements deemed to be of a low physical demand, the computing device preferably ends the motion tracking procedure with fewer flags than for other movements of a higher physical demand. A user not being capable of reproducing movements of lower physical demand is normally experiencing severe problems.

Notwithstanding, the computing device preferably also uses the data of the user for deciding to end the motion tracking procedure or not. Depending on the injury (i.e. the affected limbs and/or joints) or the overall medical condition of the user, it may be expected that movements of lower physical demand cannot be reproduced correctly by the user because they involve the motion of the affected limbs and/or joints, so the user may feel well and exercise well aside from performing movements that are particularly painful to her/him in spite of being of a lower physical demand.

In some embodiments, the step of actuating further comprises the step of communicating the at least one motion-related flag; and the step of communicating further comprises: establishing a communication link with the one or more electronic devices, and the communication link comprises one or more of: a voice communication link, a video communication link, and a data communication link.

A physical therapist or doctor can directly communicate with the user by establishing this communication link and, thus, check on the user to see what is her/his condition, whether the user needs some medical assistance, etc. What is more, the therapist or doctor can supervise the activity of the user from that point onwards during the rehabilitation session.

When the computing device also instructs the user to end the motion tracking procedure, the computing preferably first provides the corresponding indications and then proceeds to communicate the data and establish the communication link.

In some embodiments, the step of actuating further comprises: storing a temporal or permanent movement ban associated with the user, the computing device not commanding to provide one or more user perceptible signals indicative of each predetermined banned movement to be performed.

When a predetermined movement resulted in the provision of one or more flags, the computing device bans said predetermined movement or other predetermined movements of a higher physical demand. If, for instance, the user was performing a predetermined movement involving the motion of one of its legs and a plurality of motion-related flags were provided, the computing device may ban said movement or other more demanding movements involving the motion of the leg, yet the computing device allows the user to perform other movements.

In some embodiments, the step of actuating further comprises: storing a temporal or permanent motion tracking ban associated with the user, the computing device not commanding to provide one or more user perceptible signals indicative of at least one predetermined movement to be performed by the user nor processing the measurements when there is a stored active motion tracking ban associated with the user of the motion tracking system.

Following the provision of signals indicative of an end of a motion tracking procedure, the computing device digitally forbids the banned user from exercising while the ban lasts in order to minimize the risk of injury. A therapist or doctor can cancel the temporal or permanent ban, in which case the computing device receives a command to cancel the ban, for instance from the one or more electronic devices.

In some embodiments, at least for two predetermined criteria the computing device counts the number of times that said at least two predetermined criteria have been unfulfilled in a same counter.

With the counting of unfulfillment of multiple predetermined criteria using a same counter the provision of flags takes place more often. In many predetermined movements, different predetermined criteria are correlated and the unfulfillment of some of them is indicative of a potential problem, therefore the counting with the same counter may result in an earlier actuation of the system.

In some embodiments, at least for one predetermined criterion the computing device resets a counter of number of times that said at least one predetermined criterion has been unfulfilled each time a different predetermined movement is to be performed by the user.

In many predetermined movements, in spite of predetermined criteria being set commonly for some of them, they unfulfillment thereof reveals the existence of a potential problem when the number of times that they are unfulfilled exceeds a predetermined threshold only when those times occurred during the same predetermined movement. Accordingly, by resetting the counter each time a different movement is to be performed, the computing device provides flags whenever the unfulfillment of the predetermined criterion or criteria takes place during the repetitions of a same predetermined movement.

In some embodiments, the step of actuating comprises the step of commanding, and the one or more user perceptible signals indicative of the corrective action to be performed by the user are based on the predetermined movement last commanded. In some of these embodiments, the one or more user perceptible signals are further based on the predetermined criteria unfulfilled.

Depending on the physical demand that the predetermined movement last commanded has associated therewith, the indications of the corrective action may be one or another. For example, in many movements one or more body members or joints are subjected to a greater physical effort than others. Therefore, when e.g. motion-related flags are provided in such movements, the corrective action can be focused on the more demanded joint, such as: apply ice on the knee, stretch the triceps, etc.

In this sense, the predetermined movement preferably has data associated therewith for quantifying the physical demand required entirely or limb-by-limb or joint-by-joint; the data may also include predetermined indications of corrective actions to be provided as user perceptible signals during an actuation by the computing device.

The predetermined criteria, or even the predetermined constraints, of the corresponding movement can further be used to determine which corrective action is more convenient when flags are provided.

In some embodiments, the step of actuating takes place when the at least one motion-related flag and/or the at least one sound-related flag have been provided for a plurality of predetermined movements exceeding a predetermined problematic movements threshold.

The computing device may only actuate when flags have been provided in a number of predetermined movements, for example two, three, or more than three movements, and said number exceeds the predetermined problematic movements threshold. In these situations, it is assumed that the flags provided are not due to the user not being able to correctly perform one or several predetermined movements but to a condition of the user that is affecting her/him during the session.

In some embodiments, processing the sound captured to detect the human gasps comprises: processing the sound captured to compute a signal-to-noise ratio; processing the computed signal-to-noise ratio to determine whether the user is gasping, the signal-to-noise ratio being computed for time periods of the sound captured in which the human voice is detected and time periods of the sound captured in which no human voice is detected. A second aspect of the disclosure relates to a method for automatically monitoring operation of a motion tracking system and actuating based on the operation, the motion tracking system comprising both a computing device, and a plurality of inertial measurement units adapted to be arranged on a body of a user, the computing device comprising both a data communications module and at least one microphone, the method comprising: commanding, by the computing device, to provide one or more user perceptible signals indicative of at least one predetermined movement to be performed by the user;

providing, by each unit of the plurality of units, measurements of body members of the user both while each unit is arranged on a respective body member and after the user has been instructed to perform the at least one predetermined movement;

processing, by the computing device, the measurements of the plurality of units to provide a motion sequence, MS, of at least some body members of the user;

triggering, by the computing device, recalibration of the plurality of inertial measurement units when the MS does not fulfill, a number of times, one or more predetermined criteria associated with a respective movement of the at least one predetermined movement;

providing, by the computing device, a motion-related flag whenever recalibration of the units is triggered a number of times exceeding a respective predetermined threshold;

processing, by the computing device, sound captured with the at least one microphone to detect human voice therein;

processing, by the computing device, the human voice to extract words therefrom;

providing, by the computing device, a sound-related flag each time: at least some of the extracted words are present in a predetermined dictionary, and/or a number of times at least one of the extracted words that is present in the predetermined dictionary exceeds a respective predetermined threshold; and

actuating, by the computing device, when both at least one motion-related flag and at least one sound-related flag have been provided, and the step of actuating comprising at least one of the following:
commanding to provide one or more user perceptible signals indicative of the at least one motion-related flag provided and/or indicative of a corrective action to be performed by the user based on the at least one motion-related flag provided; and
communicating the at least one motion-related flag provided to one or more electronic devices.

Motion-related flags can be due to an erroneous arrangement of the inertial measurement units on the body of the user, or faulty units.

Units arranged on the user in an erroneous form comprise placing the different units on the correct body members according to the arrangement expected by the computing device (e.g., unit 1 shall be arranged on the right thigh, unit 2 shall be arranged on the right ankle, and unit 3 shall be arranged on the right foot, etc.), but with incorrect orientations or positions on the body members (e.g. unit 1 is arranged upside-down, unit 2 is arranged rotated 90° degrees, unit 1 is arranged on a side of the thigh instead of the front thereof, etc.). Said erroneous arrangement likewise comprises placing the different units on incorrect body members according to the arrangement expected by the computing device (e.g. unit 1 shall be arranged on the right upper arm but is arranged on the right lower arm, unit 2 shall be arranged on the chest but is arranged on the right upper arm, etc.) and with or without incorrect orientations or positions on the body members.

Processing measurements of units incorrectly arranged results in the provision of MS and/or ROM that is not representative of the actual movements performed by the user. Accordingly, it is very unlikely that the MS and/or the ROM will fulfill the one or more predetermined criteria (e.g. orientations, accelerations, etc. as aforesaid). When this occurs, and preferably when a number of predetermined criteria exceeding a respective predetermined threshold is not fulfilled to avoid recalibrations when it is the user who is not reproducing the movements correctly in spite of a possibly correct unit arrangement, the computing device triggers a recalibration of the units. The user, in turn, has to move the units in accordance with a predetermined calibration technique which includes particular expected motion from the units to calibrate the sensing units thereof.

When multiple recalibrations are triggered (something that occurs when after each recalibration the user tries to perform the commanded movements but the one or more predetermined criteria are fulfilled and, thus, the computing device again triggers the recalibration of the units), a motion-related flag is provided, which is indicative of the aforesaid problems.

Forcing the user to stop the physical exercising and recalibrate the units tends to disappoint the user and make the user frustrated with the motion tracking system. In such situations, the user occasionally becomes aggressive and swears. So, by processing the voice thereof as captured by the at least one microphone, extracting the words pronounced by the user, and comparing them with words in a predetermined dictionary that includes swear words or words associated with frustration and disappointment, the computing device determines whether the user is losing her/his patience and provide sound-related flags.

The human voice detection and word extraction are conducted with digital sound processing techniques known in the art and which are not part of the scope of the present disclosure; for instance, but without limitation, voice activity detection (VAD), fatigue detection, and natural language processing (NLP) techniques known in the art can be used, such as:
Greeley, H. P. & Friets, E. & Wilson, J. P. & Raghavan, S. & Picone, Joseph & Berg, Joel. (2006). “Detecting Fatigue From Voice Using Speech Recognition”, International Symposium on Signal Processing and Information Technology. 567-571. 10.1109/ISSPIT.2006.270865;
Greeley, Harold & Berg, Joel & Friets, Eric & Wilson, John & Greenough, Glen & Picone, Joseph & Whitmore, Jeffrey & Nesthus, Thomas. (2007). “Fatigue estimation using voice analysis”. Behavior research methods. 39. 610-9. 10.3758/BF03193033;
Kuldip K. Paliwal, Kaisheng Yao, Chapter 6—“Robust Speech Recognition Under Noisy Ambient Conditions”, Human-Centric Interfaces for Ambient Intelligence, Academic Press, 2010, Pages 135-162, ISBN 9780123747082;
Dharm Singh Jat, Anton Sokamato Limbo, Charu Singh, Chapter 6—“Voice Activity Detection-Based Home Automation System for People With Special Needs”, Intelligent Speech Signal Processing, Academic Press, 2019, Pages 101-111, ISBN 9780128181300; and
Ling Cen, Fei Wu, Zhu Liang Yu, Fengye Hu, Chapter 2—“A Real-Time Speech Emotion Recognition System and its Application in Online Learning”, In Emotions and Technology, Academic Press, 2016, Pages 27-46, ISBN 9780128018569;

all of which are incorporated by reference in their entirety herein.

The techniques can rely on different types of processing, like detection the presence or absence of speech and/or detection of words by e.g. spectrogram generation and processing, detection of changes in energy, or spectral distances, or cepstral distances, named entity recognition, tokenization, stemming and lemmatization, natural language generation, etc.

If the user keeps exercising when the computing device triggers the recalibrations many times, the probability of the user performing the movements incorrectly increases and the risk of getting injured also increases, and thus actuation is necessary when there are both motion-related flags and sound-related flags. The actuation can be the communication of the motion-related flags to electronic devices preferably associated with a therapist or doctor, or provide indications about the problem or the corrective action that may be taken (indications such as rearrange the units on different body members, check again the orientation of the units, etc.).

In some embodiments, the step of actuating takes place when a time elapsed between the provision of the at least one sound-related flag and the provision of the at least one motion-related flag is less than a predetermined time threshold.

In some embodiments, the step of actuating comprises the step of commanding, and the one or more user perceptible signals indicative of the corrective action to be performed by the user comprise indications of how each unit of the plurality of units shall be arranged on the body of the user.

The indications remind the user how the units shall be arranged for tracking the commanded predetermined movements.

Additionally, since the recalibration of the units is triggered when the MS and/or the ROM do not fulfill one or more predetermined criteria (preferably when two or more predetermined criteria as aforesaid), the measurements processed by the computing device may reveal how the units are arranged and, thus, where the problem is. This can be achieved, for instance, with a procedure as described in international application no. PCT/EP2020/074811, which is incorporated by reference in its entirety herein. Therefore, in those cases in which the computing device determines how the units may have been arranged on the user, the computing device can provide indications on how to rearrange each unit that is incorrectly arranged, thereby making it simpler for the user to correct the arrangement of the units thereon.

In some embodiments, the step of actuating comprises the step of communicating, and the step of communicating the at least one monition-related flag further comprises communicating: a flag indicative of a faulty motion tracking system, and/or the at least one sound-related flag.

The triggering of multiple recalibrations can be indicative of faulty units in the motion tracking system. The communication of this flag notifies the operators in charge of performing maintenance tasks on the system, and even a replacement of the motion tracking system can be scheduled in this manner. The sound related-flag(s) may also be communicated for informing the therapist or doctor about the ongoing problems.

In some embodiments, the step of actuating further comprises the step of communicating, and the step of communicating further comprises: establishing a communication link with the one or more electronic devices, and the communication link comprises one or more of: a voice communication link, a video communication link, and a data communication link.

In some embodiments, the step of actuating comprises the step of commanding, and the one or more user perceptible signals indicative of the corrective action to be performed by the user comprise indications of an end of a motion tracking procedure when both one or more motion-related flags and one or more sound-related flags have been provided. In some of these embodiments, the one or more motion-related flags provided exceeds a predetermined motion-related flag threshold. In some of these embodiments, the one or more sound-related flags provided exceeds a predetermined sound-related threshold.

In some of these embodiments, the indications of the end of the motion tracking procedure are provided in the form of the one or more user perceptible signals also based on data of the user and/or data of the predetermined movement last commanded to the user when the computing device performs the step of actuating.

In some embodiments, the step of actuating further comprises: storing a temporal or permanent motion tracking ban associated with the user, the computing device not commanding to provide one or more user perceptible signals indicative of at least one predetermined movement to be performed by the user nor processing the measurements when there is a stored active motion tracking ban associated with the user of the motion tracking system.

In some embodiments, at least for two predetermined criteria the computing device counts the number of times that said at least two predetermined criteria have been unfulfilled in a same counter.

In some embodiments, at least for one predetermined criterion the computing device resets a counter of number of times that said at least one predetermined criterion has been unfulfilled each time a different predetermined movement is to be performed by the user.

In some embodiments, the step of actuating takes place when the at least one motion-related flag and/or the at least one sound-related flag have been provided for a plurality of predetermined movements exceeding a predetermined problematic movements threshold.

Similar advantages as those described for the first aspect of the disclosure are also applicable to this aspect of the disclosure.

A third aspect of the disclosure relates to a data processing apparatus comprising at least one processor adapted to perform a method according to the first or second aspect of the disclosure.

A fourth aspect of the disclosure relates to a motion tracking system comprising: a plurality of inertial measurement units adapted to be arranged on a body of a user, and to provide measurements of body members of the user; and a computing device comprising a data communications module and at least one microphone, the computing device being adapted to execute respective steps of a method according to embodiments of the first or second aspect of the disclosure.

The motion tracking system is capable of automatically monitoring the operation thereof and the activity of the user, and actuate based on the same so as to reduce the risk of injuries and do without the supervision of a physical therapist or doctor unless completely necessary.

A fifth aspect of the disclosure relates to a computer program product that has instructions which, when executed by a computing device, cause the computing device to perform a method according to the first or second aspect of the disclosure.

Upon running the computer program product on one or more processors of the computing device, the computing device monitors the operation of a motion tracking system and the activity of the user of the system, and actuate based on the same.

In some embodiments, the computer program product is embodied on a non-transitory computer-readable medium or a computer-readable data carrier has the computer program product stored thereon.

A sixth aspect of the disclosure relates to a data carrier signal carrying a computer program product according to the fifth aspect of the disclosure.

Similar advantages as those described for the first and second aspects of the disclosure are also applicable to the third, fourth, fifth, and sixth aspects of the disclosure.

### BRIEF DESCRIPTION OF THE DRAWINGS

To complete the description and in order to provide for a better understanding of the disclosure, a set of drawings is provided. Said drawings form an integral part of the description and illustrate embodiments of the disclosure, which should not be interpreted as restricting the scope of the disclosure, but just as examples of how the disclosure can be carried out. The drawings comprise the following figures:

FIG. 1 diagrammatically shows a motion tracking system in accordance with embodiments.

FIG. 2 shows a user of a motion tracking system with inertial measurement units arranged thereon in accordance with embodiments.

FIGS. 3 and 4 show the user of FIG. 2 while performing two different predetermined movements in accordance with embodiments.

FIGS. 5-7 show graphs representing the unfulfillment of predetermined criteria and provision of flags in accordance with embodiments.

FIG. 8 shows an exemplary gasp detection by methods and systems in accordance with embodiments.

### DESCRIPTION OF WAYS OF CARRYING OUT THE DISCLOSURE

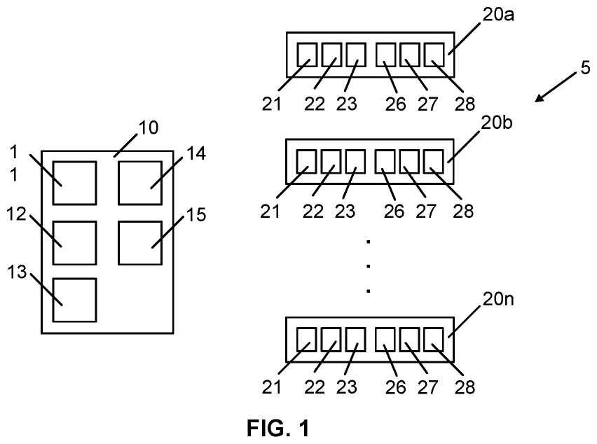

FIG. 1 diagrammatically shows a motion tracking system 5 in accordance with embodiments. The motion tracking system 5 includes a plurality of inertial measurement units 20a-20n, IMUs, and a computing device 10, which may be e.g. a tablet, a mobile phone, a personal computer, etc.

Each IMU 20a-20n is adapted to be arranged on the body of a user so that the measurements provided by each IMU 20a-20n can be processed by the computing device 10, thereby providing a motion sequence of the user. The IMUs 20a-20c may be attached to the body members of the user in a number of ways, for instance using straps, Velcro, etc. Each IMU 20a-20n is a motion tracker that includes one or more sensing devices selected from e.g. an accelerometer 21, a gyroscope 22 and a magnetometer 23. In the embodiment of FIG. 1, each IMU 20a-20n includes all three sensing devices 21-23, but in other embodiments the IMUs only include an accelerometer 21 and a gyroscope 22, for instance. Preferably, all IMUs 20a-20n include the same sensing devices 21-23.

The IMUs 20a-20n further include at least one processor 26, at least one memory 27, and a first communications module 28 for transmitting radiofrequency signals to the computing device 10 and which include e.g. advertisement packages, data packets with identification data (e.g. one or more identities, keys, etc.), data packets with measurements of the sensing device(s) 21-23, etc. When no wireless communications connections are established with the computing device 10, the radiofrequency signals of the IMUs 20a-20n include advertisement packages for indicating their presence and that they are active. Once the wireless communications connections are established (using a technology and protocol known by a skilled person, for instance but without limitation, Bluetooth and Bluetooth Low Energy communications, cellular network communications such as GSM, UMTS or LTE, wireless LAN communications, etc.) with the computing device 10, the radiofrequency signals of the IMUs 20a-20n may include identification data and/or the measurements. An antenna for radiating electromagnetic waves is provided as part of the first communications module 28. Preferably, the same first communications modules 28 also enable the IMUs 20a-20n to receive data from the computing device 10 upon capturing electromagnetic waves with the antenna.

Preferably, at least one processor 26 of the IMUs 20a-20n runs a sensor fusion algorithm for processing the measurements of the sensing devices 21-23 within the respective sensor. The sensor fusion algorithm is intended to enhance the raw measurements of the sensing devices by correcting errors thereof due to drifts of the sensing devices and, thus, outputs processed measurements that are to be transmitted to the computing device 10.

The computing device 10 includes at least one processor 11, at least one memory 12, and a second communications module 13 for at least receiving data. The second communications module 13 includes at least one antenna whereby electromagnetic waves may be captured, and then processed by the at least one processor 11.

The computing device 10 also includes at least one microphone 14, and preferably includes a screen 15. On said screen 15 the computing device 10 is capable of showing instructions and/or information to the intended user about the operation of the system 5, for example predetermined movements that are to be performed by an intended user of the motion tracking system 5, indications of a correspondence between IMUs 20a-n and body members of the user for arrangement thereof, potential problems detected, provision of flags, etc. To this end, the computing device 10 stores, in the at least one memory 12, data indicative of a correspondence between IMUs and users assigned to those IMUs, and also data relative to the physical exercises, i.e. predetermined movements, of intended users. Any of these data can be transmitted to and/or received from another electronic device thanks to the second communications module 13. For example, a therapist is able to receive the feedback at a computing device in a hospital so as to monitor the evolution of the person. Based on the feedback received, the therapist is able to adjust the difficulty of the movement(s), the number of repetitions thereof, prescribe new movements, etc. so that the person may further exercise using the motion tracking system 5. Further, in addition to the screen 15, which provides visual feedback, the computing device 10 may also include further visual output means (e.g. LEDs, animations), audio output means (e.g. loudspeakers), vibrating means (e.g. a vibrator), etc. for providing user perceptible signals in the form of sounds, vibration, animated graphics, etc. The computing device 10 controls the information provided by the screen 15 or by any other user presenting means by way of digital commands sent to the particular device(s).

The system 5 could additionally or alternatively include other user presenting means like the ones mentioned above and which the computing device 10 can control by way of wired or wirelessly-transmitted commands.

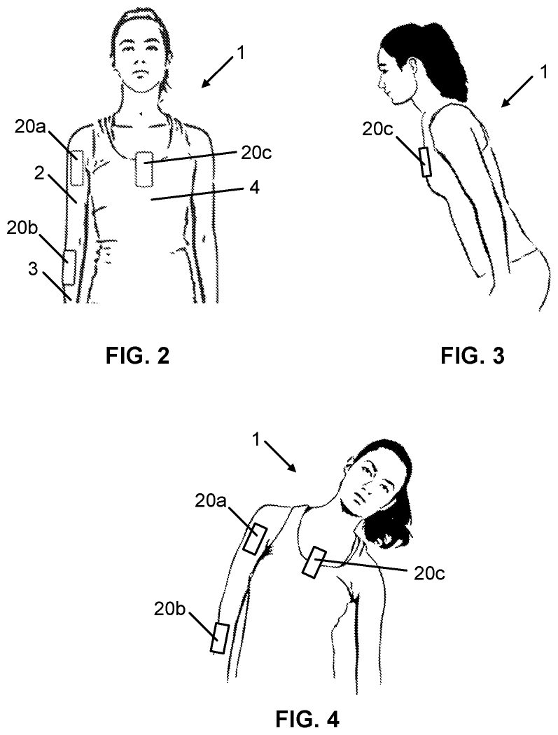

FIG. 2 shows a user 1 of a motion tracking system with IMUs 20a-20c arranged thereon in accordance with embodiments. The user 1 is standing still.

A first IMU 20a is arranged on a right upper arm 2, a second IMU 20b is arranged on a right lower arm 3, and a third IMU 20c is arranged on a chest 4.

As known in the art, the user 1 arranges the IMUs 20a-20c in this way according to information provided by a computing device of the motion tracking system about where and how (i.e. position and angle) each IMU 20a-20c is to be arranged; alternatively, the computing device may only inform the user 1 about which body members 2-4 shall have an IMU 20a-20c arranged thereon and how, the user 1 places the IMUs 20a-20c accordingly, and then by way of an automated calibration procedure or with user input means the IMU-body member correspondence is registered by the computing device. Concerning the former, the computing device may command the provision of user perceptible signals indicative of one or more predetermined calibration movements that the user 1 is to perform to determine which IMU 20a-20c is on each body member 2-4 based on the measurements provided by the IMUs 20a-20c during reproduction of the movement, whereas in the latter forms can be shown to the user 1 to specify what IMU 20a-20c is on what body member 2-4.

FIG. 3 shows the user 1 of FIG. 2 while performing a predetermined movement in which, while standing still, an upper body is to be moved forward while maintaining the hips static relative to the floor. FIG. 4 shows the same user 1 while performing a predetermined movement in which, while standing still, an upper body is to be moved to one side while maintaining the hips static relative to the floor.

These predetermined movements and additional ones or other ones can be part of a physical rehabilitation procedure whereby the user 1 intends to improve her/his physical condition.

The physical rehabilitation typically includes a number of predetermined movements, each with its respective number of repetitions. When the rehabilitation is to be conducted with a motion tracking system, each predetermined movement also includes respective one or more predetermined criteria (including one or more predetermined constraints) so that people can undergo a rehabilitation procedure without direct supervision of a therapist or doctor. The type and number of predetermined movements, the numbers of repetitions, and the predetermined criterion or criteria are assigned to each particular user 1 based on the physical condition thereof and the recovery plan the user has to follow. All these parameters can change with the number of sessions taken by the user 1. In this sense, the computing device stores or retrieves data with all the parameters necessary to instruct the user 1 to perform the predetermined movements and monitor the activity of the user 1 while exercising.

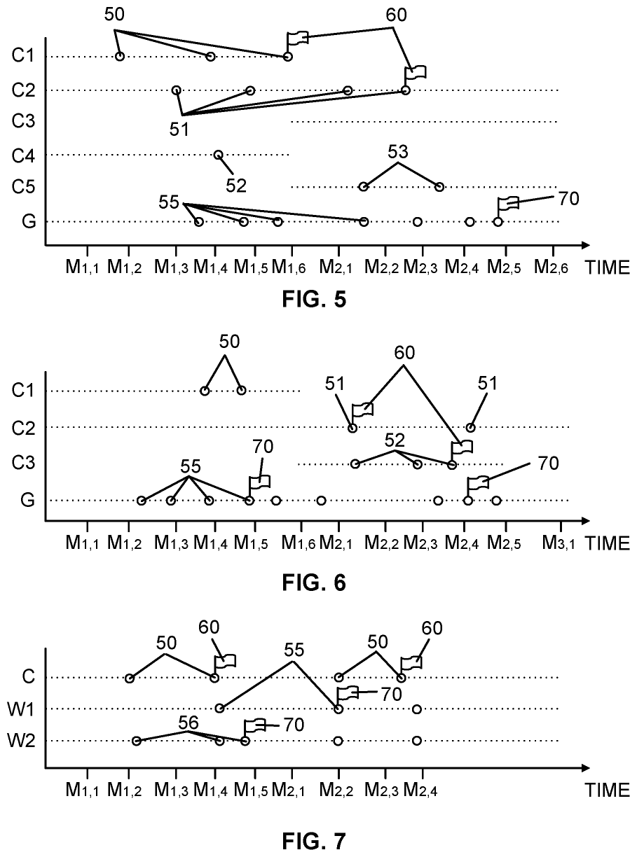

FIGS. 5-7 show graphs representing the unfulfillment of predetermined criteria and provision of flags 60, 70 in accordance with embodiments.

The horizontal axes of the graphs represent time. In the horizontal axis of each graph are marks showing when a user of a motion tracking system ends a repetition of a predetermined movement, the coding M_{I,J} is used, where I stands for the number of predetermined movement and J stands for the repetition of the l-th predetermined movement; for example, the mark M_{2,4} represents the time when the user has finished the fourth repetition of the second predetermined movement. Some sessions include a single predetermined movement, whereas other sessions include two, three, four or even more predetermined movements. Each predetermined movement typically includes a plurality of repetitions.

In the vertical axes are represented different predetermined criteria. The circles represent each time a predetermined criterion is unfulfilled as the session advances over time. For the sake of the illustration only, the dotted lines represent for which predetermined movements each predetermined criterion is defined. In this sense, some predetermined movements may have a same predetermined criterion when it comes to ROM, starting position, etc.

Referring to FIG. 5, four predetermined criteria C1, C2, C4 and G are set for a first predetermined movement M₁, and four predetermined criteria C2, C3, C5 and G are set for a second predetermined movement M₂. A predetermined threshold may be set for each particular predetermined criterion, and/or a predetermined threshold may be set for two or more particular predetermined criteria as will be described with reference to FIG. 6.

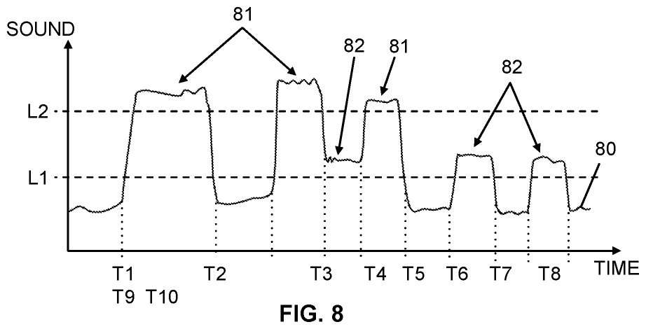

The criterion G illustrated in FIGS. 5 and 6 relates to the determination of the user gasping. Each time the computing device determines that the user gasps (in the manner described in FIG. 8 for example) a circle marker 55 is illustrated in the graphs.

In the example of FIG. 5, the criterion G has a threshold of four gasps for a same predetermined movement performed by the user, whereas in the example of FIG. 6 the value of the threshold is the same but is per session.

As it can be seen in the graph of FIG. 5, the fourth gasp that is detected takes place during the second repetition of the second predetermined movement M_{2,2}, and the three previous gasps have taken place during repetitions of the first predetermined movement M₁. Hence, a sound-related flag 70 is not provided until M_{2,5}, when four gasps have occurred during M₂.

Regarding the first criterion C1 set for M₁ only, is unfulfilled 50 three times. The respective threshold is three per session (that is to say, the computing device counts all the times the criterion is unfulfilled 50 irrespective of whether the unfulfillments 50 occur during a single movement or different movements), and at the third unfulfillment 50 a motion-related flag 60 is provided.

The second criterion C2, set for both M₁ and M₂, is unfulfilled 51 four times. The respective threshold is four per session, and at the fourth unfulfillment 51 a motion-related flag 60 is provided.

The third criterion C3, set for M₂ only, is not unfulfilled and, thus, no motion-related flag 60 is provided due to C3.

The fourth criterion C4, set for M₁ only, is unfulfilled 52 once. The respective threshold is three per movement and no motion-related flag 60 is provided due to C4.

The fifth criterion C5, set for M₂ only, is unfulfilled 53 twice. The respective threshold is four per session and no motion-related flag 60 is provided due to C5.

The motion tracking system actuates when at least one motion-related flag 60 and at least one sound-related flag 70 are provided. Therefore, when the first motion-related flag 60 is provided, no action is taken. Since six repetitions are set for M₁, after the sixth repetition thereof the commanded predetermined movement becomes M₂, with six repetitions as well. By the time the second motion-related flag 60 is provided no sound-related flag 70 has been provided yet, so there is no actuation by the computing device of the system. At M_{2,5} the sound-related flag 70 is provided, and at that moment either the motion-related flags 60 are communicated to one or more electronic devices and/or some feedback is provided to the user. Despite that, no change in the predetermined movement is made and the sixth repetition of the second predetermined movement is completed by the user. In this sense, it may occur that the actuation of the computing device is a silent communication to the therapist or doctor, and the user is not informed of any problematic situations or actuations based on the same.

In relation to FIG. 6, criteria C1-C3 and G are set. The user is instructed to perform three different predetermined movements M₁-M₃.

In this case, a predetermined threshold is set in cumulative form in respect of criteria C1 and C2, even if C1 is not set for M₂ and M₃ but C2 is. Said threshold has a value of three per session (it could be per movement too). Despite this cumulative predetermined threshold, one or both of the criteria C1 and C2 may also have respective predetermined thresholds, either per session or per movement.

The first criterion C1 is unfulfilled 50 twice. The respective threshold is three per movement, but no motion-related flag 60 is provided due to this threshold.

Before the user starts M₂, a sound-related flag 70 is provided due to the number of gasps of the user. The lack of a motion-related flag 60 in M₁ does not trigger any actuation.

In M₂, the second criterion C2 is unfulfilled 51 twice. In spite of its respective threshold being five per session, a motion-related flag 60 is provided due to the cumulative predetermined threshold. This is so because the first criterion C1 was unfulfilled 50 twice during M₁ and at the first unfulfillment 51 of the second criterion C2 during M₂ the cumulative threshold was met. If the cumulative threshold had been set per movement, the unfulfillment 51 of the second criterion C2 would have to be during M₁ to produce the provision of the motion-related flag 60.

Depending on the user, the computing device can actuate as soon as the motion-related flag 60 is provided owing to the existence of the sound-related flag 70. For example, when the user is an elderly person, the computing device may want to take some action to avoid the risk of the user getting injured. In this example the user is a person without a severe medical condition and, thus, there is no actuation with just one flag 60, 70 of each, but at least two motion-related flags 60, 70, or at least three flags 60, 70 of one kind and one or more of the other 70, 60. In some other examples, the actuation may take place when one flag 60, 70 of each kind is provided but with a time difference between the two that is less than a predetermined time threshold.

The third criterion C3, set for M₂ only, is unfulfilled 52 three times. The respective threshold is three per session, and at the third unfulfillment 52 (during M_{2,4}) a motion-related flag 60 is provided.

A second sound-related flag 70 is provided during M_{2,5}. As aforesaid, the threshold for criterion G in the example of FIG. 6 is four per session, thus the second sound-related flag 70 is provided although the first of the four unfulfillments 55 took place during M_{1,6}. As it can be appreciated, once a flag 60, 70 is provided, a respective counter of times that a criterion has been unfulfilled is reset.

The existence of two motion-related flags 60 and two sound-related flags 70 triggers an actuation, in this case the computing device commands the provision of signals to instruct the user to skip to another predetermined movement in spite of the second predetermined movement having eight repetitions set for the ongoing session.

In FIG. 7, there are three predetermined criteria C, W1 and W2. The criterion C is unfulfilled 50 whenever the computing device triggers a recalibration of the IMUs. The criterion W1 is unfulfilled 55 whenever the computing device detects that the user has pronounced a particular word that is present within a predetermined dictionary, whereas the criterion W2 is unfulfilled 56 whenever the computing device detects that the user has pronounced different words present within the predetermined dictionary. Accordingly, every time the criterion W1 is unfulfilled 55 the criterion W2 is also unfulfilled 56 since it relates to the pronunciation of an offending word.

The rehabilitation session is programmed to have four different predetermined movements, with ten repetitions of each.

The criterion C is unfulfilled 50 four times, and its predetermined threshold is two per session. The criterion W1 is unfulfilled 55 three times, and its predetermined threshold is two per session. The criterion W2 is unfulfilled 56 five times, and its predetermined threshold is three per session

While the user performs repetitions of M₁, two recalibrations are triggered because multiple predetermined constraints of M₁ that the user had to comply with were not met, which may be indicative of an incorrect unit arrangement or units that are not calibrated, i.e. the sensing devices thereof measure with an error that is deemed to be excessive. A motion-related flag 60 is provided. Shortly thereafter, a sound-related flag 70 is provided owing to the criterion W2. The computing device instructs the user to skip to M₂ in case the problems are not due to the calibration of the units or the arrangement thereof but to incorrect reproduction of M₁ by the user.

During M₂, again two recalibrations are triggered and, hence, a motion-related flag 60 is again provided. By the time the second motion-related flag 60 is provided, another sound-related flag 70 has been provided due to the criterion W1. So, at M_{2,4}, following the provision of the motion-related flag 60, the computing device signals the end of the motion tracking procedure and instructs the user to stop all activity, thereby not continuing with any one of the second, third and fourth predetermined movements.

FIG. 8 shows an exemplary gasp detection by methods and systems in accordance with embodiments.

A graph is illustrated in which sound 80 captured by at least one microphone of a computing device is plotted versus time. In this example, two level thresholds L1, L2 are set for detecting gasps, which preferably are dynamically adjustable but can be constant as well and selected based on experimental tests or assumptions (e.g. expected speech and noise power respective exceeding and not exceeding certain power levels). The sound 80 and level thresholds are represented in dB, but other units may be used too.

As sound 80 is captured, the computing device processes it so as to detect human voice in it. Based on the detection or non-detection, speech and noise levels are determined, and preferably the level thresholds L1, L2 are set based on said speech and noise levels. In order to increase the confidence in the determination of the speech level, when the computing device considers that human voice is present in certain parts of the sound 80, it runs word extraction techniques on it to establish whether said part in fact is speech.

Accordingly, the first level threshold L1 is set or adjusted based on the expected sound level when there is no human voice; said threshold is above said level by a predetermined factor. All sound level not exceeding the first level threshold L1 is deemed to be a baseline sound level.

Likewise, the second level threshold L2 is set or adjusted based on the expected sound level when there is human voice, and preferably when said human voice corresponds to speech; said threshold is below said level by a predetermined factor. All sound level exceeding the second level threshold L2 is deemed to be speech, and more particularly words 81; preferably the computing device confirms that they are words 81 by running the word extraction techniques.

As for the sound level between the first and second level thresholds L1, L2, the same is deemed to be gasps 82.

Said word and gasp detection can rely on the level of the sound 80 itself and the procedure described above, or on the level of the sound 80 in terms of signal-to-noise ratio, SNR. In the latter case, the level thresholds L1, L2 are SNR level thresholds. The SNR is greater when the user is speaking, and becomes lower when the user does not, but the SNR is still greater when the user is gasping (and especially between words 81, e.g. between T4 and T5) than the SNR when there user is neither speaking nor gasping.

In the graph represented, silence and, thus, noise is captured from an initial time instant up to T1. From T1 to T2 one or several words 81 are pronounced by the user. From T2 to T3 there is no speech nor gasps because the sound 80 does not exceed the first threshold level L1. From T3 to T4 a word 81 is pronounced by the user, and after that and up to T5 there is a gasp 82 followed by another word between T5 and T6. From T6 to T7 there is silence. And, again, between T7 and T8 and between T9 and T10, the user gasps 82 two more times.

In this text, the term “comprises” and its derivations (such as “comprising”, etc.) should not be understood in an excluding sense, that is, these terms should not be interpreted as excluding the possibility that what is described and defined may include further elements, steps, etc.

On the other hand, the invention is obviously not limited to the specific embodiment(s) described herein, but also encompasses any variations that may be considered by any person skilled in the art (for example, as regards the choice of materials, dimensions, components, configuration, etc.), within the general scope of the invention as defined in the claims.

1. A method for automatically monitoring operation of a motion tracking system and actuating based on the operation, the motion tracking system comprising both a computing device, and a plurality of inertial measurement units adapted to be arranged on a body of a user, the computing device comprising both a data communications module and at least one microphone, the method comprising: commanding, by the computing device, to provide one or more user perceptible signals indicative of at least one predetermined movement to be performed by the user; providing, by each unit of the plurality of units, measurements of body members of the user both while each unit is arranged on a respective body member and after the user has been instructed to perform the at least one predetermined movement; processing, by the computing device, the measurements of the plurality of units to provide a motion sequence, MS, of at least part of at least some body members of the user; storing, by the computing device and associated with both the user and a movement of the at least one predetermined movement, the MS and/or a range of motion, ROM, of one or more of the some body members of the user, the ROM being computed by the computing device based on the MS; processing, by the computing device, the MS and/or the ROM to check fulfillment thereof of one or more predetermined criteria associated with a respective movement of the at least one predetermined movement and counting, by the computing device, a number of times that each of the one or more predetermined criteria are unfulfilled; providing, by the computing device, a motion-related flag each time a counted number of times does not meet a respective predetermined threshold; processing, by the computing device, sound captured with the at least one microphone to detect human voice therein; processing, by the computing device, the sound captured to detect human gasps therein based on the detected human voice; providing, by the computing device, a sound-related flag when it determines that the user has gasped a number of times exceeding a respective predetermined threshold; and actuating, by the computing device, when both at least one motion-related flag and at least one sound-related flag have been provided, wherein the step of actuating comprises at least one of the following: commanding to provide one or more user perceptible signals indicative of the at least one motion-related flag provided and/or indicative of a corrective action to be performed by the user based on the at least one motion-related flag provided; and communicating the at least one motion-related flag provided to one or more electronic devices.

2. A method for tracking a motion of a subject, the method comprising:
(a) providing a motion tracking system comprising:
(i) a computing device;
(ii) a plurality of inertial measurement units, wherein an inertial measurement unit of the plurality of inertial measurement units is arranged on a body member of the subject; and
(iii) at least one microphone;
(b) providing one or more indications of at least one movement to the subject;
(c) measuring a plurality of signals associated with the at least one movement using at least (1) the plurality of inertial measurement units and (2) the at least one microphone; and
(d) using the computing device to process the plurality of signals measured in (c), thereby tracking the motion of the subject.

3. The method of claim 2, further comprising processing the plurality of signals measured in (c) to provide a motion sequence or a range of motion of the one or more body members of the subject.

4. The method of claim 2, further comprising using the computing device to determine a presence of a motion related flag based at least in part on the plurality of signals measured in (c).

5. The method of claim 4, further comprising using the computing device to count a number of occurrences of the motion related flag.

6. The method of claim 5, wherein the computing device processes at least one signal of the plurality of signals is used to detect a number of gasps of the user.

7. The method of claim 7, wherein the detection of the number of gasps of the user is performed by determining a number of sounds which meet a signal to noise level threshold.

8. The method of claim 6, further comprising determining that greater than a threshold number of gasps or a threshold number of motions have been detected; and indicating to the user a corrective action based on the predetermined movement.

9. The method of claim 8, further comprising determining from the range of motion or the motion sequence whether one or more predetermined criteria associated with the predetermined movement are met.

10. The method of claim 9, wherein the one or more predetermined criteria comprise at least one of: the motion sequence not fulfilling one or more predetermined constraints set for each predetermined movement commanded; the range of motion being lower than a range of motion previously stored for the same user and for the same predetermined movement; and an elapsed time for the one or more body members in the motion sequence to be in the initial position of the predetermined movement exceeding a predetermined time threshold.

11. The method of claim 9, wherein the one or more predetermined criteria at least comprises the predetermined criterion of the range of motion being lower than a range of motion previously stored, and wherein said criterion comprises: an average of the range of motion being lower than an average of the range of motion previously stored, and a difference between the averages exceeding a predetermined average range of motion threshold; and/or the range of motion of the respective movement of the one or more predetermined movements in a plurality of repetitions of the respective movement being lower than the average of the range of motion previously stored.

12. The method of claim 9, further comprising recalibrating of the plurality of inertial measurement units when the motion sequence does not fulfill, a number of times, one or more of the predetermined criteria associated with the predetermined movement.

13. The method of claim 9, wherein indicating of the corrective action comprises indications of how each unit of the plurality of units shall be arranged on a body of the user.

14. The method of claim 9, wherein indicating of the corrective action comprises indicating that the motion tracking system is faulty.

15. The method of claim 9, wherein the user is instructed to perform the predetermined motion and the measured signals comprise time elapsed from when the instruction is given.

16. The method of claim 9, wherein indicating of the corrective action comprises indicating that the motion tracking procedure is complete.

17. The method of claim 9, further comprising storing a temporal or permanent motion tracking record of the user.

18. The method of claim 17, wherein the temporal or permanent motion tracking record comprises a ban associated with the user; and wherein the user is not permitted to operate the motion tracking system when there is a stored active motion tracking ban associated with the user.

19. The method of claim 9, further comprising establishing a communication link between the computing system and one or more electronic devices, wherein the communication link comprises one or more of: a voice communication link, a video communication link, and a data communication link.

20. The method of claim 9, wherein the at least one predetermined criteria comprise at least two predetermined criterion.

21. The method of claim 20, wherein the at least two predetermined criterion are counted independently.
